(19) United States
(12) Patent Application Publication
(10) Pub. No.: US 2007/0219830 A1
(43) Pub. Date: Sep. 20, 2007

(54) System and method of remote care on-line monitoring

(76) Inventors: Adrian F. Warner, Delafield, WI (US); Crispian L. Sievenpiper, Waukesha, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/384,190

(22) Filed: Mar. 16, 2006

(51) Int. Cl. A61B 5/00

(52) U.S. Cl. 705/3; 600/301

(57) ABSTRACT

A system and a method by which a clinician may monitor and communicate with a plurality of remotely located patients. This monitoring may be facilitated using a variety of communication protocols. Modular treatments and institutionally created standards are combined with clinician-provided input and decisions to provide the patient an individualized treatment regimen.

### FIELD OF THE INVENTION

The present invention pertains to the field of biotelemetry and remote patient care.

### BACKGROUND OF THE INVENTION

The rising cost of health care is of concern to patients and hospital administrators alike. One way to reduce the cost of medical treatment is to reduce the number of days spent in the hospital as there are large fundamental costs associated with each night spent in a hospital bed. As a result, there has been a continued push towards home centered and/or remote off-site patient treatment and/or convalescence. This presents significant challenges for health care providers due to the lack of personal interaction and the lack of a suitably dedicated IT infrastructure. A particular difficulty with home centered or remote off-site treatment is the lack of a patient-care provider interaction which results in the inability of the health care provider to observe the subtle indicators that are relevant to evaluate the current treatment protocol or any adverse reactions to current treatment. Another challenge of remote care is providing 24/7 care to patients that may live alone. Furthermore, remote off-site care presents new challenges in overcoming the distance between the patient and the health care provider upon the detection of an adverse reaction or event.

The field of remote patient monitoring, or biotelemetry, has developed modern technologies and techniques for the remote physiological monitoring of patients. The current modern communication infrastructure including, but not limited to, WIFI, cellular, TCP/IP, and VOIP platforms is suitable to support health care provider-patient communications. These platforms may be used individually or in combination to provide a medical treatment network supporting voice and data transmission. Therefore, it is desirable in the field of biotelemetry to provide a system that is capable of integrating these technologies to build a cohesive care delivery system that facilitates the provision of remote patient care. It is also desirable in the field of biotelemetry to have a system of care that prioritizes patient needs to facilitate the efficient provision of care to remote patients. SUMMARY OF THE INVENTION

Remote patient care could be better supported through the use of modern communications infrastructure and monitoring devices that are combined with new forms of traditional diagnostic instrumentation and control. The provision of remote care is facilitated by integrating these technologies to build a cohesive care delivery system that better enables the monitoring of patient vital parameters, medication, and treatment while supporting full bidirectional communication.

A patient monitoring device kept on the patient facilitates the patient monitoring and communication back to a centralized computer network. This network provides for the transmission of treatment and medication information which can better advance treatment delivery and compliance through treatment schedule reminders and access to treatment information. This works in conjunction with the monitoring of vital parameters to increase the ability to monitor and/or detect adverse reactions to medication.

The communications network allows for the bidirectional transmission of voice and data information between the remote patient and the centralized network. The increased communication capability helps the health care provider to bridge the distance between the health care provider and the remote patient by providing the health care provider with a new means to confer with the patient and to confer the subtle observations that promote patient monitoring.

A complex array of institutional and health care provider created rules within a patient treatment file is used to regulate the monitoring of patient vital parameters for signals that are outside of specified limits. The treatment of remote patients is facilitated by escalation levels that are associated with exceeding rule limits in addition to the use of more traditional and simplistic rate level or quantity triggers. In an embodiment of the present invention, multiple levels of escalation allow for a more controlled proactive response in kind to the level of escalation leading to a variety of anticipated conditions. The gradation of patient escalation facilitates the ability to queue, route, and triage patients for the efficient provision of treatment to patients at remote locations.

Various other features, objects and advantages of the invention will be made apparent from the following description taken together with the drawings.

### BRIEF DESCRIPTION OF THE DRAWINGS

The drawings illustrate the best mode presently contemplated of carrying out the invention.

In the drawings:

FIG. 1 is a schematic diagram of an embodiment of the remote patient care system of the present invention;

FIG. 2 is a flowchart of the provision of a patient prescription for use in the present invention;

FIG. 3 is a schematic diagram of the escalation of patient condition and prioritization of patient treatment of the present invention.

### DETAILED DESCRIPTION OF THE INVENTION

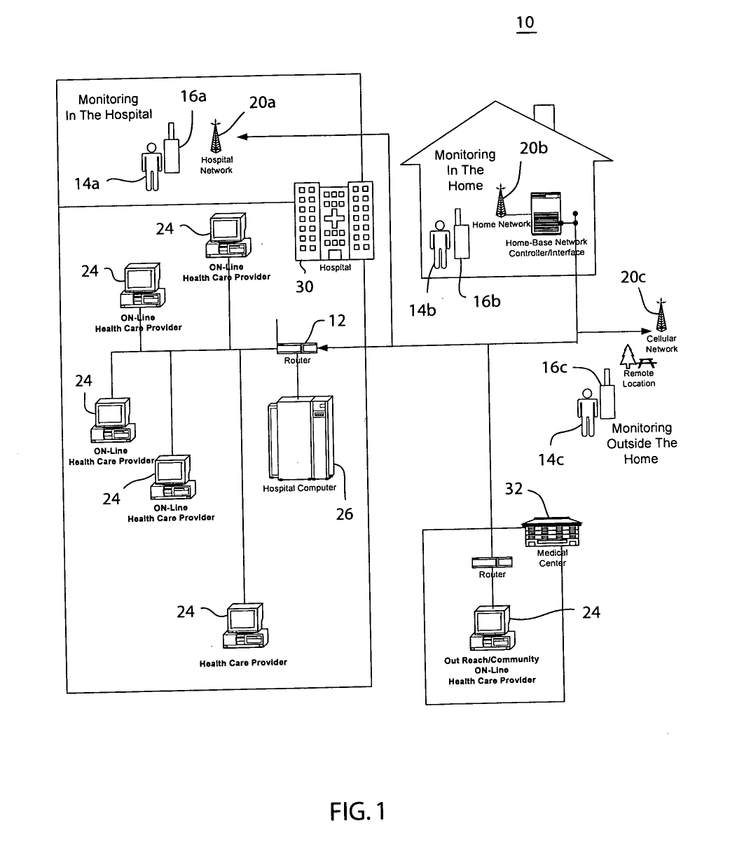

As shown in FIG. 1, a remote care network 10 provides data and voice communication between a centralized computer network 12 and a variety of remote patients 14. The remote patients may consist of a patient at a remote location within the hospital 14a, a remote patient in their home 14b, or a remote patient outside their home 14c. Each patient is equipped with a monitoring device 16 that is kept on the person and specifically programmed for each patient 14a-14c, respectively. The monitoring device 16 communicates to a local network 20 which may be a hospital network 20a, a home network 20b, or an area network 20c outside the home or hospital. These networks 20 may comprise a variety of network means and/or protocols including but not limited to WIFI, cellular, TCP/IP, and VOIP. In an embodiment of the present invention, the patient monitoring device 16 connects to the local network 20 via the optimal available connection means. Once the monitoring device 16 has connected to the remote care network 10, bidirectional voice and data transmission is possible with any of a variety of on-line health care providers 24 and/or information databases 26 which may be available at a hospital 30 or another medical center 32 that is a part of the network 10.

The bidirectional voice and data transmission of the remote care network 10 helps the system of the present invention to overcome limitations associated with the provision of care to remotely monitored patients. The ability of the patient and/or the clinician to initiate contact with the other allows for the remotely monitored patient to maintain a sense of being cared for that the patient would experience if admitted in a hospital. This makes the patient more comfortable in the remote setting for treatment and/or convalescence and aids in patient treatment compliance. Additionally, the bidirectional communication of the remote care network of the present invention enables the clinician to contact the patient to obtain so-called “soft” information such as patient responsiveness, reasoning ability, and mood that aids a clinician in evaluating the progression of a treatment. The clinician may also use the communication abilities of the remote care network as a confirmation of a detected manual or automatic escalation alert, or may provide a first stage of response by the clinician to the notification of an escalation alert.

The patient monitoring device 16 remains operational despite the lack of a sufficient local network 20. In the absence of a proper communications signal, the monitoring device 16 buffers and stores its measured data. The monitoring device 16 then transmits all of the stored information to the centralized computer network 12 when a suitable local network 20 is available. This allows the patient to leave a local network 20 without fear of losing medical data or monitoring device 16 functionality. Additionally, monitoring device 16 may include circuitry (not shown) for the evaluation and optimization of the local network 20 platform. For example, a patient may already have a WIFI connection in his home, or may be in a location where free WIFI connectivity is provided. In these instances, while a cellular connection would also probably be available, it would come at a much greater connectivity cost. Therefore, the circuitry would select to use the WIFI connection. Alternatively, the patient may be in a location where only expensive cellular connectivity is available, therefore the circuitry would choose to store and buffer the medical data, only connecting cellularly at standardized intervals to transmit recorded patient data, or connect upon the action of the patient or the clinician to connect.

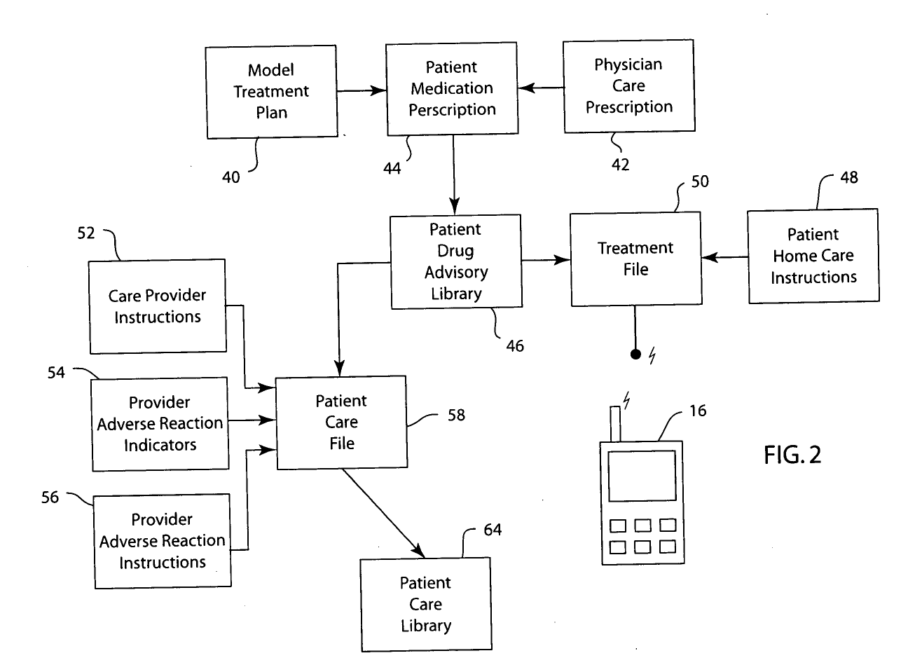

Referring now to FIG. 2, a patient that has been designated for remote care is first brought into the remote care network by creating a patient treatment file that combines a base physician approved model treatment plan 40 that has been adopted by the institution with an individualized care prescription 42 as written by a physician to create a patient medication prescription 44. This prescription 44 is screened with the patient drug advisory library 46 to check for any potential complication from the proposed treatment. Patient home care instructions 48, which may include additional information regarding medication and/or treatment, are added to create a patient treatment file 50 which is downloaded to the patient monitoring device 16. The patient treatment file 50 downloaded to the monitoring device 16 in an embodiment of the present invention, provides the patient with information about the medication and treatment, a medication treatment schedule, and medication dosage reminders.

The patient medication prescription 44 and the patient home care instructions 48 within the patient's treatment file 50 include useful treatment information for the patient which is aimed at patient treatment compliance. Embodiments of the present invention may include alarms that are scheduled based on the prescription that reminds the patient of when, what, and how to take the medication. These alarms may be stored as information files that are replayed as audio files and/or may be displayed as a textual display on the monitoring device 16. An embodiment of the present invention may also include a means for detecting, identifying, and confirming the medications that have been prescribed to the patient. This may be facilitated by, but is not limited to, an identification means such as a bar code label that is placed on the medication bottle and a bar code scanner integrated with the monitoring device. As a further aspect of an embodiment of the present invention, a notification of a medication event may be sent back to the centralized computer network for recording in a database, or so that the clinician may use the communications features of the remote care network to confirm that the patient has complied with the medication alert.

On the health care provider side of the remote care network, after the prescription 44 has been screened with the patient drug advisory library 46, the prescription is combined with care provider instructions 52, provider adverse reaction indicators 54, and provider adverse reaction instructions 56 to create a patient care file 58. The care provider instructions 52 may include additional directions and/or reminders of tests to be performed by the health care provider. The provider adverse reaction indicators 54 comprise a series of escalation rules comprising a combination of boolean statements, value range limits, and/or patient parameter level triggers. The provider adverse reaction instructions 56 correspond with the adverse reaction indicators identified in 54 to provide instructions to the health care provider for the action to be taken in the event of a triggering of an adverse reaction indicator.

The patient care file 58 is saved in a patient care on-line library 64 within the remote care network 10 and is used to facilitate the analysis of incoming patient physiological data transmitted from the patient monitoring device 16.

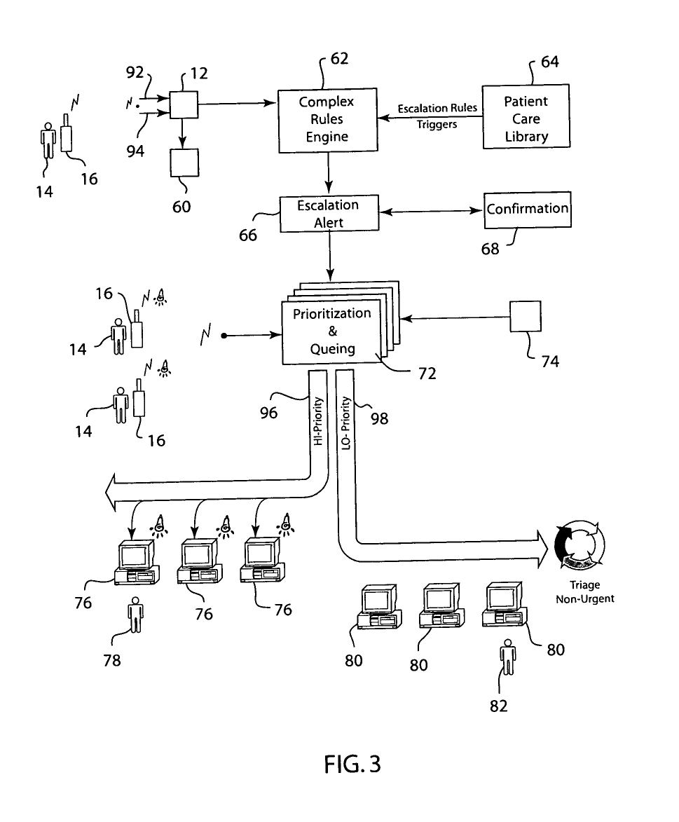

FIG. 3 depicts a schematic diagram of the operation of the system of the present invention. The system includes the transmission of physiological signals, the interpretation of patient escalation, and the prioritization of patient treatment. A patient 14 is being monitored by a monitoring device 16. This monitoring device 16 is collecting and transmitting parametric data 92 and other treatment events 94 to a centralized computer network 12. These parametric data 92 may be collected by modular biomedical instruments (not shown) that are added to the monitoring device 16 to tailor the monitoring to the individual patient. These parametric data 92 may also be collected by stand-alone biomedical instruments that are independently connected to a patient (not shown), but communicate with the monitoring device 16 and transmit measured data to monitoring device 16.

These modular components may comprise, but are not limited to, temperature, ECG, pulse oximetry, respiration rate, blood pressure, and blood glucose monitoring devices. The addition of a GPS system to monitoring device 16 would allow the clinician to track a patient's position and would also facilitate locating the patient if emergency medical treatment is needed. Treatment events 94 may include patient activities, times at which medications were taken, or patient communications.

The centralized computer network 12 records the received data in a data storage 60. The data storage 60 provides a useful log for the full disclosure recordation of patient treatment. The event log combined with the physiological data can provide a useful tool for data analysis and patient trend tracking. The data is also transmitted to a complex rules engine 62 where the parametric data and recorded events are compared to the patient escalation rules and triggers in the patient care file 58 from the patient care on-line library 64. If the patient's parametric data and/or treatment events are outside of any escalation rule limits or escalation trigger levels, an escalation alert 66 is created. This alert may then be sent through a confirmation process 68 to determine the validity of the escalation alarm. Upon confirmation, the escalation alerts 66 of all monitored patients are combined with any alerts that have been manually activated by any patients 14 using a designated alert key (not shown) on the monitoring device 16.

The combined alerts are analyzed using a database of institutional prioritization rules 74 to separate the alerts into high priority alerts and low priority alerts. The high priority alerts 96 will then be transmitted to an on-line health care provider 76 with the patient care file so that the on-line clinician 78 may take the appropriate actions. The low priority alerts 98 are sent to on-line health care providers 80 where the low priority alerts are triaged and queued based upon the institutional prioritization rules 74. These alerts are then reviewed and responded to by an on-line clinician 82 in the order that they are queued.

The prioritization and queuing feature of an embodiment of the present invention adds to the efficiency of the remote patient care system. As the remote patient care system may be providing care to patients that are physically removed from a health care facility by up to a substantial distance. Therefore, this distance must be taken into account when evaluating the order in which additional on-site patient care is provided. The prioritization and queuing feature enables the remote patient care system to automatically prioritize and queue the received incoming patient escalation alerts based upon the severity of the escalation and the need for additional care by a clinician. Under this system, a patient with a severe escalation alert will receive treatment and a clinician's attention before a lower priority or severity alert is addressed. This optimizes the clinician's ability to treat the patient, especially when large and time consuming distances must be traversed to provide the additional care to a remote patient.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Various alternatives and embodiments are contemplated as being within the scope of the following claims particularly pointing out and distinctly claiming the subject matter regarded as the invention.

1. An electronic probe apparatus for testing electronic circuits, said apparatus includes an epoxy probe card having probe wires mounted therein and having an electrically insulating surface and further electronic components mounted on said electrically insulating surface, said components being connected to said probe wires by electrically conducting lines formed on said electrically insulating surface of said probe card, said probe card further includes a metallic stiffener ring being part of said epoxy probe card, at least one of said probe wires that is carrying a common or ground potential is electrically connected by an electrically conducting line formed on said electrically insulating surface to said metallic stiffener ring.

2. An electronic probe apparatus for testing electronic circuits, said apparatus includes an epoxy probe card having probe wires mounted therein and having an electrically insulating surface and further having electronic components mounted on said electrically insulating surface, said components being connected to said probe wires by electrically conducting lines located on said electrically insulating surface of said probe card, wherein at least one of said probe wires is connected to an electrically conducting layer extending over or under said electrically insulating surface.

3. The electronic probe card apparatus of claim 1, wherein said at least one probe wire is connected to an electrically conducting layer having been applied to a bottom of said insulating surface of said epoxy probe.

4. The electronic probe apparatus including an epoxy probe card, said epoxy probe card is mounted on a circuit board and further including at least one special probe mounted on said circuit board after said epoxy probe card has been mounted on said circuit board, said special probe has a metallic tip attached to an insulating shank, said metallic tip is used for contacting the circuit to be tested.
